(19) United States
(12) Patent Application Publication
(10) Pub. No.: US 2026/0257206 A1
(43) Pub. Date: Sep. 3, 2026

(54) METHOD FOR PREPARING A CATALYST CONTAINING AN ACTIVE NICKEL PHASE AND A NICKEL-COPPER ALLOY

(71) Applicant: IFP Energies nouvelles, RUEIL-MALMAISON (FR)

(72) Inventors: Malika BOUALLEG, RUEIL-MALMAISON CEDEX (FR); Laetitia JOTHIE, RUEIL-MALMAISON CEDEX (FR)

(73) Assignee: IFP Energies nouvelles, RUEIL-MALMAISON (FR)

(21) Appl. No.: 18/995,617

(22) PCT Filed: Jul. 11, 2023
(86) PCT No.: PCT/EP2023/069134

§ 371 Date: Jan. 16, 2025

(30) Foreign Application Priority Data
Jul. 21, 2022 (FR) FR2207484

(51) Int. Cl. B01J 23/755; B01J 6/00; B01J 21/04; B01J 35/45

(52) U.S. Cl. CPC B01J 23/755; B01J 6/001; B01J 21/04; B01J 35/45

(57) ABSTRACT

A process for preparing a catalyst comprising nickel and copper and a porous alumina support, comprising the following steps:
a) bringing the alumina support into contact with a solution containing a nickel precursor and a first organic compound comprising at least one carboxylic acid function;
b) drying at a temperature of less than 250° C., then calcining at a temperature of between 250° C. and 550° C.;
c) bringing the calcined catalyst precursor obtained at the end of step b) into contact with a solution containing a nickel precursor, a copper precursor and a second organic compound comprising at least one carboxylic acid function;
d) drying at a temperature of less than 250° C.

### TECHNICAL FIELD

The present invention relates to a supported metal catalyst based on nickel and copper intended particularly for the hydrogenation of unsaturated hydrocarbons, and more particularly for the selective hydrogenation of polyunsaturated compounds or the hydrogenation of aromatics.

### STATE OF THE ART

Catalysts for the selective hydrogenation of polyunsaturated compounds or for the hydrogenation of aromatic compounds are generally based on metals from group VIII of the Periodic Table of Elements, such as nickel. The metal is in the form of nanometric metal particles deposited on a support which may be a refractory oxide. The content of metal from Group VIII, the optional presence of a second metal element, the size of the metal particles and the distribution of the active phase in the support and also the nature and the pore distribution of the support are parameters which may have an influence on the performance of the catalysts.

The rate of the hydrogenation reaction is governed by several criteria, such as the diffusion of the reactants toward the surface of the catalyst (external diffusional limitations), the diffusion of the reactants in the porosity of the support toward the active sites (internal diffusional limitations) and the intrinsic properties of the active phase, such as the size of the metal particles and the distribution of the active phase within the support.

For the purpose of obtaining better catalytic performance, notably a better selectivity and/or activity, it is known in the prior art to use additives of organic compound type for the preparation of metal catalysts for selective hydrogenation. For example, the application FR 2 984 761 discloses a process for the preparation of a selective hydrogenation catalyst comprising a support and an active phase comprising a metal from Group VIII, said catalyst being prepared by a process comprising a step of impregnation of the support with a solution containing a precursor of the metal from Group VIII and an organic additive, more particularly an organic compound exhibiting from one to three carboxylic acid functions, a step of drying the impregnated support and a step of calcination of the dried support in order to obtain the catalyst.

Moreover, the promotion of a nickel-based catalyst has frequently been proposed in order to improve performance levels in selective hydrogenation. For example, it is known from U.S. Pat. No. 5,208,405 to use a catalyst based on nickel and silver for the selective hydrogenation of C₄-C₁₀ diolefins. Furthermore, it is known to promote nickel, predominantly present, with metals from Group IB, in particular gold (FR 2 949 077) or tin (FR 2 949 078). Document FR 3 011 844 discloses a catalyst for the implementation of a selective hydrogenation process comprising a support and an active metallic phase deposited on the support, the active metallic phase comprising copper and at least one metal out of nickel or cobalt in a Cu:(Ni and/or Co) mole ratio greater than 1.

Finally, prior to the employment of such catalysts and the use thereof in a hydrogenation process, a step of reducing treatment in the presence of a reducing gas is carried out so as to obtain a catalyst comprising an active phase at least partially in metallic form. This treatment makes it possible to activate the catalyst and to form metal particles. This treatment may be carried out in situ or ex situ, that is to say after or before the catalyst is charged to the hydrogenation reactor.

Document FR3099390A1 discloses a catalyst comprising nickel and copper, in a proportion of 1% to 50% by weight of nickel element relative to the total weight of the catalyst, in a proportion of 0.5% to 15% by weight of copper element relative to the total weight of the catalyst, and an alumina support, said catalyst being obtained via a preparation process comprising the following steps:
a) the alumina support is brought into contact with at least one solution containing at least one nickel precursor;
b) the alumina support is brought into contact with at least one solution containing at least one nickel precursor and at least one copper precursor;
c) the alumina support is brought into contact with at least one solution containing at least one organic compound comprising at least one carboxylic acid function, or at least one alcohol function, or at least one ester function, or at least one amide function, or at least one amine function,
it being understood that:
   steps a), b) and c) are carried out separately in any order; or
   steps a) and c) are carried out simultaneously, step b) being carried out either before the combination of steps a) and c) or after;
   steps b) and c) are carried out simultaneously, step a) being carried out either before the combination of steps b) and c) or after;
d) at least one step of drying the catalyst precursor obtained at the end of steps a) to c) is carried out at a temperature of less than 250° C.;
e) a step of reducing the catalyst precursor obtained at the end of step d) is carried out by bringing said precursor into contact with a reducing gas at a temperature of greater than or equal to 150° C. and less than 250° C.

Continuing their research in the field of catalysts for selective hydrogenation of polyunsaturated compounds or hydrogenation of aromatic compounds, the applicant has now identified that it is possible to prepare a particularly active catalyst by carrying out a specific preparation process wherein a metallic phase based on nickel and copper is added in the presence of a particular organic additive to the catalyst after the deposition of the precursor of the active phase based on nickel and also in the presence of a particular organic additive.

Without wishing to be bound by any theory, it has been observed by the applicant that, during the preparation of the catalyst, carrying out a step of bringing the catalyst into contact with a solution simultaneously containing a copper-based metal precursor, a nickel-based metal precursor and a particular organic additive, followed by a step of drying and reducing in the presence of a reducing gas at low temperature (greater than or equal to 150° C. and less than 200° C.) makes it possible to obtain a nickel-copper alloy (in reduced form) which unexpectedly makes it possible to greatly improve the reducibility of the nickel active phase on the support, said nickel active phase being supplied in a step prior to the formation of the nickel-copper alloy (in reduced form). The preparation process according to the invention thus makes it possible to carry out a step of reducing the metal elements in the presence of a reducing gas at lower temperatures and shorter reaction times than those commonly used in the prior art. Advantageously, the use of less severe operating conditions than in the prior art makes it possible to directly carry out the reduction step within the reactor in which it is the aim to carry out the hydrogenation of aromatic compounds. However, the addition of nickel and copper after the addition of the active nickel phase inevitably leads to the enlargement of the nickel particles and therefore to a substantial loss of activity. This is why the applicant has identified that the addition of a particular organic additive, at contents much higher than those used during impregnation of the active nickel phase, makes it possible to limit the enlargement of the nickel particles or even to avoid it altogether.

### Subjects of the Invention

One subject of the present invention is a process for preparing a catalyst comprising nickel and copper, in a proportion of 1% to 50% by weight of nickel element relative to the total weight of the catalyst, and in a proportion of 0.5% to 15% by weight of copper element relative to the total weight of the catalyst, and a porous alumina support, the size of the nickel particles in the catalyst, measured in oxide form, being less than 5 nm, which process comprises at least the following steps:
a) the alumina support is brought into contact with at least one solution containing at least one first nickel precursor and at least one first organic compound comprising at least one carboxylic acid function to obtain a first catalyst precursor;
b) the first catalyst precursor obtained at the end of step a) is dried at a temperature of less than 250° C. and then the dried first catalyst precursor is calcined at a temperature of between 250° C. and 550° C. to obtain a calcined catalyst precursor;
c) the calcined catalyst precursor obtained at the end of step b) is brought into contact with at least one solution containing at least one second nickel precursor, at least one copper precursor and at least one second organic compound comprising at least one carboxylic acid function to obtain a second catalyst precursor;
d) the second catalyst precursor obtained at the end of step c) is dried at a temperature of less than 250° C.

According to one or more embodiments, the mole ratio between said organic compound introduced in step a) and the nickel element also introduced in step a) is between 0.01 and 5.0 mol/mol.

According to one or more embodiments, the mole ratio between said organic compound introduced in step c) and the nickel element also introduced in step c) is between 0.02 and 5 mol/mol.

According to one or more embodiments, the mole ratio between the nickel introduced during steps a) and c) and the copper introduced during step c) is between 0.5 and 5 mol/mol.

According to one or more embodiments, the ratio between the mole ratio between the second organic compound and the nickel introduced in step c) and the mole ratio between the first organic compound and the nickel introduced in step a) is greater than 1.5.

According to one or more embodiments, steps a) and b) are carried out at least twice before carrying out step c).

According to one or more embodiments, the first organic compound of step a) and the second organic compound of step c) are chosen from oxalic acid, malonic acid, glycolic acid, lactic acid, tartronic acid, citric acid, tartaric acid, pyruvic acid and levulinic acid.

According to one or more embodiments, the first organic compound of step a) and the second organic compound of step c) are identical.

According to one or more embodiments, the copper precursor is chosen from copper acetate, copper acetylacetonate, copper nitrate, copper sulfate, copper chloride, copper bromide, copper iodide and copper fluoride.

According to one or more embodiments, the first nickel precursor and/or the second nickel precursor are/is nickel nitrate, nickel chloride, nickel acetate or nickel hydroxycarbonate.

According to one or more embodiments, said process also comprises a step e) wherein the catalyst obtained at the end of step d) is calcined at a temperature of between 250° C. and 550° C.

According to one or more embodiments, said process also comprises a step f) wherein the catalyst obtained at the end of step d), optionally obtained at the end of step e), is reduced by bringing said catalyst into contact with a reducing gas at a temperature of greater than or equal to 150° C. and less than 250° C.

Another subject according to the invention relates to a catalyst obtained via the preparation process according to the invention.

Another subject according to the invention relates to a process for the selective hydrogenation of polyunsaturated compounds containing at least 2 carbon atoms per molecule, contained in a hydrocarbon feedstock having a final boiling point below or equal to 300° C., which process being carried out at a temperature of between 0° C. and 300° C., at a pressure of between 0.1 and 10 MPa, at a hydrogen/(polyunsaturated compounds to be hydrogenated) mole ratio of between 0.1 and 10 and at an hourly space velocity of between 0.1 and 200 h⁻¹ when the process is carried out in the liquid phase, or at a hydrogen/(polyunsaturated compounds to be hydrogenated) mole ratio of between 0.5 and 1000 and at an hourly space velocity of between 100 and 40 000 h⁻¹ when the process is carried out in the gas phase, in the presence of a catalyst according to the invention or obtained according to the preparation process according to the invention.

Another subject according to the invention relates to a process for the hydrogenation of at least one aromatic or polyaromatic compound present in a hydrocarbon feedstock having a final boiling point of less than or equal to 650° C., said process being carried out in the gas phase or in the liquid phase, at a temperature of between 3° and 350° C., at a pressure of between 0.1 and 20 MPa, at a hydrogen/(aromatic compounds to be hydrogenated) mole ratio of between 0.1 and 10 and at an hourly space velocity of between 0.05 and 50 h⁻¹, in the presence of a catalyst according to the invention or obtained according to the preparation process according to the invention.

### DETAILED DESCRIPTION OF THE INVENTION

### 1. Definitions

Subsequently, the groups of chemical elements are given according to the CAS classification (CRC Handbook of Chemistry and Physics, published by CRC Press, editor-in-chief D. R. Lide, 81st edition, 2000-2001). For example, group VIII (or VIIIB) according to the CAS classification corresponds to the metals of columns 8, 9 and 10 according to the new IUPAC classification.

In the present description, according to the IUPAC convention, “micropores” are understood to mean the pores having a diameter of less than 2 nm, i.e. 0.002 μm; “mesopores” are understood to mean the pores having a diameter of greater than 2 nm, i.e. 0.002 μm, and less than 50 nm, i.e. 0.05 μm, and “macropores” are understood to mean the pores having a diameter of greater than 50 nm, i.e. 0.05 μm.

The total pore volume is measured by mercury porosimetry according to the standard ASTM D4284-92 with a wetting angle of 140°, for example using an Autopore III™ model device of the Micromeritics™ brand.

The BET specific surface area is measured by nitrogen physisorption according to the standard ASTM D3663-03, a method described in the work by Rouquerol F., Rouquerol J. and Singh K., “Adsorption by Powders & Porous Solids: Principles, Methodology and Applications”, Academic Press, 1999.

The median mesopore diameter is also defined as being the diameter such that all the pores, among the combined pores constituting the mesopore volume, with a size of less than this diameter constitute 50% of the total mesopore volume determined by mercury porosimetry intrusion.

The term “size of the nickel particles” is understood to mean the diameter of the nickel crystallites in oxide form. The diameter of the nickel crystallites in oxide form is determined by X-ray diffraction, from the width of the diffraction line located at the angle 2θ=43° (that is to say, along the crystallographic direction [200]) using the Scherrer relationship. This method, used in X-ray diffraction on polycrystalline samples or powders, which links the full width at half maximum of the diffraction peaks to the size of the particles, is described in detail in the reference: Appl. Cryst. (1978), 11, 102-113, “Scherrer after sixty years: A survey and some new results in the determination of crystallite size”, J. I. Langford and A. J. C. Wilson. The content of nickel and copper is measured by X-ray fluorescence.

In the present description, the term “to comprise” is synonymous with (means the same thing as) “to include” and “to contain”, and is inclusive or open-ended and does not exclude other elements that are not stated. It is understood that the term “to comprise” includes the exclusive and closed term “to consist of”. In addition, in the present description, the term “substantially” corresponds to an approximation of ±10%, preferably of ±5%, very preferably of ±2%, of a reference value, such as a distance, a velocity, a flow rate, a content of compounds, a temperature, a pressure, etc.

### 2. Process for the Preparation of the Catalyst

The steps of said preparation process are described in detail below.

Step a) Bringing the Support into Contact with a First Nickel Precursor and a First Organic Compound

The deposition of the first nickel precursor and of the first organic compound comprising at least one carboxylic acid function, on said support, in accordance with the implementation of step a), can be carried out by dry impregnation or excess impregnation, or also by deposition—precipitation, according to methods well known to a person skilled in the art.

Preferably, said step a) is carried out by dry impregnation, which consists in bringing the catalyst support into contact with a solution containing at least the first nickel precursor and at least one first organic compound comprising a carboxylic acid function, the volume of the solution of which is between 0.25 and 1.5 times the pore volume of the support to be impregnated.

Said step a) is preferably carried out by impregnation of the support consisting, for example, in placing said support in contact with at least one solution, which is aqueous or organic (for example methanol or ethanol or phenol or acetone or toluene or dimethyl sulfoxide (DMSO)) or indeed consists of a mixture of water and of at least one organic solvent, containing at least the first nickel precursor at least partially in the dissolved state and at least a first organic compound comprising a carboxylic acid function, or else in bringing said support into contact with at least one colloidal solution of at least one nickel precursor, in the oxidized form (nanoparticles of oxide, of oxy (hydroxide) or of hydroxide of the nickel) or in the reduced form (metal nanoparticles of the nickel in the reduced state) and of at least one first organic compound comprising a carboxylic acid function. Preferably, the solution is aqueous. The pH of this solution could be modified by the optional addition of an acid or of a base.

Preferably, said first nickel precursor is introduced in aqueous solution, for example in nitrate, carbonate, acetate, chloride or oxalate form, in the form of complexes formed by a polyacid or an acid alcohol and its salts, in the form of complexes formed with acetylacetonates or in the form of any other inorganic derivative soluble in aqueous solution, which is brought into contact with said support. Preferably, use is advantageously made, as first nickel precursor, of nickel nitrate, nickel chloride, nickel acetate or nickel hydroxycarbonate. Very preferably, the first nickel precursor is nickel nitrate.

The concentration of nickel in solution is adjusted depending on the pore volume of the support still available so as to obtain, for the supported catalyst, a nickel content of between 1% and 50% by weight of nickel element relative to the total weight of the catalyst, more preferentially between 2% and 40% by weight and even more preferentially between 3% and 35% by weight and even more preferentially 5% and 28% by weight.

Said first organic compound comprising at least one carboxylic acid function may be a saturated or unsaturated aliphatic organic compound or an aromatic organic compound. Preferably, the saturated or unsaturated aliphatic organic compound comprises between 1 and 9 carbon atoms, preferably between 2 and 7 carbon atoms. Preferably, the aromatic organic compound comprises between 7 and 10 carbon atoms, preferably between 7 and 9 carbon atoms.

Said first saturated or unsaturated aliphatic organic compound or said aromatic organic compound comprising at least one carboxylic acid function may be chosen from monocarboxylic acids, dicarboxylic acids, tricarboxylic acids or tetracarboxylic acids.

Advantageously, the first organic compound comprising at least one carboxylic acid function is chosen from ethanedioic acid (oxalic acid), propanedioic acid (malonic acid), pentanedioic acid (glutaric acid), hydroxyacetic acid (glycolic acid), 2-hydroxypropanoic acid (lactic acid), 2-hydroxypropanedioic acid (tartronic acid), 2-hydroxypropane-1,2,3-tricarboxylic acid (citric acid), 2,3-dihydroxybutanedioic acid (tartaric acid), 2-oxopropanoic acid (pyruvic acid) or 4-oxopentanoic acid (levulinic acid).

Advantageously, the mole ratio of the first organic compound introduced in step a) to the nickel element also introduced in step a) is between 0.01 and 5.0 mol/mol, preferably between 0.05 and 2.0 mol/mol, more preferentially between 0.1 and 1.5 mol/mol and more preferentially still between 0.3 and 1.2 mol/mol.

### Step b) Drying and Calcining

The first precursor of the catalyst, obtained at the end of step a), is then dried at a temperature of less than 250° C., preferably of between 15° C. and 180° C., more preferentially between 30° C. and 160° C., more preferentially still between 50° C. and 150° C. and in an even more preferential way between 70° C. and 140° C., for a period of time typically of between 0.5 hour and 12 hours and more preferably for a period of time of between 0.5 hour and 5 hours. Longer periods of time are not ruled out but do not necessarily contribute an improvement.

The drying step can be carried out by any technique known to a person skilled in the art. It is advantageously performed under an inert atmosphere or under an oxygen-containing atmosphere or under a mixture of inert gas and oxygen. It is advantageously carried out at atmospheric pressure or at reduced pressure. Preferably, this step is carried out at atmospheric pressure and in the presence of air or nitrogen.

After drying, the first dried catalyst precursor is calcined at a temperature of between 250° C. and 600° C., preferably between 350° C. and 550° C., for a period typically of between 0.5 and 24 hours, preferably for a period of between 0.5 and 12 hours, and even more preferably for a period of between 0.5 and 10 hours, preferably under an inert atmosphere or under an oxygen-containing atmosphere. Longer periods of time are not ruled out but do not necessarily contribute an improvement.

Step c) Bringing the Calcined Catalyst Precursor into Contact with a Copper Precursor, a Second Nickel Precursor and a Second Organic Compound

The deposition of the nickel, of the copper and of the second organic compound comprising at least one carboxylic acid function, on the calcined catalyst precursor obtained at the end of step b), can be carried out by dry impregnation or excess impregnation, or also by deposition—precipitation, according to methods well known to a person skilled in the art.

Preferably, said step c) is carried out by dry impregnation, which consists in bringing the calcined catalyst precursor into contact with a solution comprising, preferably consisting of, at least one nickel precursor, at least one copper precursor and at least one second organic compound comprising at least one carboxylic acid function, the volume of the solution of which is between 0.25 and 1.5 times the pore volume of the support to be impregnated.

Said step c) is preferentially carried out by simultaneous impregnation of the calcined catalyst precursor obtained at the end of step b) consisting for example in bringing said calcined catalyst precursor into contact with at least one solution, which is aqueous or organic (for example methanol or ethanol or phenol or acetone or toluene or dimethyl sulfoxide (DMSO)) or else consists of a mixture of water and at least one organic solvent, comprising, preferably consisting of, at least one second nickel precursor at least partially in the dissolved state, at least one copper precursor at least partially in the dissolved state and one second organic compound comprising at least one carboxylic acid function, or else bringing said calcined catalyst precursor into contact with at least one colloidal solution comprising, preferably consisting of, at least one nickel precursor and one copper precursor in oxidized form (nanoparticles of oxide, of oxy (hydroxide) or of hydroxide of nickel and copper) or in reduced form (metallic nanoparticles of nickel and copper in the reduced state) and at least one second organic compound comprising at least one carboxylic acid function. Preferably, the solution is aqueous. The pH of this solution may be modified by the optional addition of an acid or of a base.

Preferably, said second nickel precursor and the copper precursor are introduced in aqueous solution.

When the second nickel precursor is introduced in aqueous solution, use is advantageously made of the second nickel precursor in the nitrate, carbonate, acetate, chloride, hydroxide, hydroxycarbonate, oxalate, sulfate or formate form, in the form of complexes formed by a polyacid or an acid alcohol and its salts, in the form of complexes formed with acetylacetonates, in the form of tetrammine or hexamine complexes, or else in the form of any other inorganic derivative soluble in aqueous solution, which is brought into contact with said catalyst precursor. Preferably, nickel nitrate, nickel hydroxide, nickel carbonate, nickel chloride or nickel hydroxycarbonate is advantageously used as second nickel precursor. Very preferably, the second nickel precursor is nickel nitrate, nickel carbonate or nickel hydroxide.

When the copper precursor is introduced in aqueous solution, a copper precursor in mineral or organic form is advantageously used. In mineral form, the copper precursor can be chosen from copper acetate, copper acetylacetonate, copper nitrate, copper sulfate, copper chloride, copper bromide, copper iodide or copper fluoride. Very preferably, the copper precursor salt is copper nitrate.

The second nickel precursor is advantageously supplied in step c) at a desired concentration in order to obtain on the final catalyst (i.e. obtained at the end of the drying/calcination step d) or the reduction step e) if the latter is carried out) a content of between 0.5% and 10% by weight of nickel element relative to the total weight of the final catalyst, preferably between 0.5% and 8% by weight, more preferentially between 1% and 7% by weight, even more preferentially between 1% and 5% by weight.

The amounts of the copper precursor(s) introduced into the solution according to step c) are chosen such that the total copper content is between 0.5% and 15% by weight of copper element relative to the total weight of the final catalyst (i.e. obtained at the end of the drying/calcination step d) or the reduction step e) if the latter is carried out), preferably between 0.5% and 12% by weight, preferably between 0.75% and 10% by weight, and even more preferentially between 1% and 9% by weight.

Said second organic compound comprising at least one carboxylic acid function may be a saturated or unsaturated aliphatic organic compound or an aromatic organic compound. Preferably, the saturated or unsaturated aliphatic organic compound comprises between 1 and 9 carbon atoms, preferably between 2 and 7 carbon atoms. Preferably, the aromatic organic compound comprises between 7 and 10 carbon atoms, preferably between 7 and 9 carbon atoms.

Said second saturated or unsaturated aliphatic organic compound or said aromatic organic compound comprising at least one carboxylic acid function may be chosen from monocarboxylic acids, dicarboxylic acids, tricarboxylic acids or tetracarboxylic acids.

Advantageously, the second organic compound comprising at least one carboxylic acid function is chosen from ethanedioic acid (oxalic acid), propanedioic acid (malonic acid), pentanedioic acid (glutaric acid), hydroxyacetic acid (glycolic acid), 2-hydroxypropanoic acid (lactic acid), 2-hydroxypropanedioic acid (tartronic acid), 2-hydroxypropane-1,2,3-tricarboxylic acid (citric acid), 2,3-dihydroxybutanedioic acid (tartaric acid), 2-oxopropanoic acid (pyruvic acid) or 4-oxopentanoic acid (levulinic acid).

Advantageously, the mole ratio of the second organic compound introduced in step c) to the nickel element also introduced in step c) is between 0.02 and 5 mol/mol, preferably between 0.1 and 3 mol/mol, more preferentially between 0.2 and 2 mol/mol and more preferentially still between 0.3 and 2 mol/mol.

Advantageously, the ratio between the mole ratio between the second organic compound and the nickel introduced in step c) and the mole ratio between the first organic compound and the nickel introduced in step a) is greater than or equal to 1.5, preferably between 2 and 5.

Advantageously, the second organic compound introduced in step c) is identical to the first organic compound introduced in step a).

### Step d) Drying

The second precursor of the catalyst, obtained at the end of step c), is then dried at a temperature of less than 250° C., preferably of between 15° C. and 180° C., more preferentially between 30° C. and 160° C., more preferentially still between 50° C. and 150° C. and in an even more preferential way between 70° C. and 140° C., for a period of time typically of between 0.5 hour and 12 hours and more preferably for a period of time of between 0.5 hour and 5 hours. Longer periods of time are not ruled out but do not necessarily contribute an improvement.

The drying step can be carried out by any technique known to a person skilled in the art. It is advantageously performed under an inert atmosphere or under an oxygen-containing atmosphere or under a mixture of inert gas and oxygen. It is advantageously carried out at atmospheric pressure or at reduced pressure. Preferably, this step is carried out at atmospheric pressure and in the presence of air or nitrogen.

### Step e) Calcination (Optional)

After drying, the catalyst obtained at the end of step d) is advantageously calcined at a temperature of between 250° C. and 600° C., preferably between 350° C. and 550° C., for a period typically of between 0.5 and 24 hours, preferably for a period of between 0.5 and 12 hours, and even more preferably for a period of between 0.5 and 10 hours, preferably under an inert atmosphere or under an oxygen-containing atmosphere. Longer periods of time are not ruled out but do not necessarily contribute an improvement.

### Step f) Reduction by a Reducing Gas (Optional)

In one embodiment according to the invention, prior to the use of the catalyst in the catalytic reactor and the implementation of a hydrogenation process, a reducing treatment step f) is carried out in the presence of a reducing gas so as to obtain a catalyst comprising nickel at least partially in the metallic form. This step is advantageously carried out in situ, that is to say after charging of the catalyst to a hydrogenation reactor. This treatment makes it possible to activate said catalyst and to form metal particles, in particular of nickel in the zero-valent state. The in situ implementation of the catalyst reducing treatment makes it possible to dispense with an additional step of passivation of the catalyst with an oxygen-bearing compound or CO₂, which is necessarily the case when the catalyst is prepared by carrying out a reducing treatment ex situ, that is to say outside the reactor used for the hydrogenation of aromatic or polyaromatic compounds. In fact, when the reducing treatment is carried out ex situ, it is necessary to carry out a passivation step in order to preserve the metallic phase of the catalyst in the presence of air (during operations of transport and charging of the catalyst to the hydrogenation reactor), then to carry out a new step of reducing the catalyst.

The reducing gas is preferably hydrogen. The hydrogen may be used pure or as a mixture (for example a hydrogen/nitrogen, hydrogen/argon or hydrogen/methane mixture). In the case where the hydrogen is used as a mixture, any proportion may be envisaged.

Said reducing treatment is carried out at a temperature above or equal to 150° C. and below 250° C., preferably between 16° and 230° C., and more preferentially between 17° and 220° C. The duration of the reducing treatment is between 5 minutes and less than 5 hours, preferably between 10 minutes and 4 hours, and even more preferentially between 10 minutes and 210 minutes.

The presence of the nickel-copper alloy at least partially in reduced form makes it possible to use operating conditions for reducing the nickel active phase which are less severe than in the prior art and thus makes it possible to carry out the reduction step directly within the reactor in which it is desired to carry out the hydrogenation of unsaturated or aromatic compounds.

Furthermore, the presence of copper in the catalyst makes it possible to preserve good activity of the catalyst and a good service life of the catalyst when the latter is placed in contact with a hydrocarbon feedstock comprising sulfur. Indeed, compared to nickel, the copper present in the catalyst more easily captures the sulfur-containing compounds included in the feedstock, which limits the irreversible poisoning of the active sites. The rise in temperature up to the desired reduction temperature is generally slow, for example set between 0.1 and 10° C./min, preferably between 0.3 and 7° C./min.

The hydrogen flow rate, expressed in I/hour/gram of catalyst precursor, is between 0.01 and 100 l/hour/gram of catalyst, preferably between 0.05 and 10 l/hour/gram of catalyst precursor and more preferably still between 0.1 and 5 l/hour/gram of catalyst precursor.

### 3. Catalyst

The preparation process according to the invention makes it possible to obtain a catalyst comprising nickel and copper, in a proportion of 1% to 50% by weight of nickel element relative to the total weight of the catalyst, and in a proportion of 0.5% to 15% by weight of copper element relative to the total weight of the catalyst, and a porous alumina support, the size of the nickel particles in the catalyst, measured in oxide form, being less than 5 nm.

Preferably, at least one portion of the nickel and the copper is in the form of a nickel-copper alloy, advantageously corresponding to the formula NiₓCu_{y} with x between 0.1 and 0.9 and y between 0.1 and 0.9.

Preferably, the nickel content included in the copper-nickel alloy is between 0.5% and 15% by weight of nickel element relative to the total weight of the catalyst, preferably between 1% and 12% by weight, and more preferentially between 1% and 10% by weight.

The size of the nickel particles, measured in oxide form, in the catalyst is less than 5 nm, more preferentially less than 4 nm, and even more preferentially less than or equal to 3 nm.

The nickel content in said catalyst is advantageously between 1% and 50% by weight relative to the total weight of the catalyst, more preferentially between 2% and 40% by weight and even more preferentially between 3% and 35% by weight and even more preferentially 5% and 25% by weight relative to the total weight of the catalyst.

The copper content is between 0.5 and 15% by weight of copper element relative to the total weight of the catalyst, preferably between 0.5 and 12% by weight, preferably between 0.75 and 10% by weight, and even more preferentially between 1 and 9% by weight.

The specific surface area of the catalyst is generally between 10 m²/g and 350 m²/g, preferably between 25 m²/g and 300 m²/g, more preferably between 40 m²/g and 250 m²/g.

The total pore volume of the catalyst is generally between 0.1 and 1 ml/g, preferably between 0.2 ml/g and 0.8 ml/g, and particularly preferably between 0.3 ml/g and 0.7 ml/g.

The active phase of the catalyst preferably does not comprise a metal from Group VIB. It notably does not comprise molybdenum or tungsten.

Said catalyst (and the support used for the preparation of the catalyst) is in the form of grains advantageously having a diameter of between 0.5 and 10 mm. The grains may have any form known to a person skilled in the art, for example the form of beads (preferably having a diameter of between 1 and 8 mm), of extrudates, of tablets or of hollow cylinders. Preferably, the catalyst (and the support used for the preparation of the catalyst) are in the form of extrudates with a diameter of between 0.5 and 10 mm, preferably between 0.8 and 3.2 mm and very preferably between 1.0 and 2.5 mm and with a length of between 0.5 and 20 mm. The “diameter” of the extrudates is intended to mean the diameter of the circle circumscribed in the cross section of these extrudates. The catalyst can advantageously be presented in the form of cylindrical, multilobe, trilobe or quadrilobe extrudates. Preferably, its shape will be trilobe or quadrilobe. The shape of the lobes will be able to be adjusted according to all the known methods of the prior art.

### 4. Support

The characteristics of the alumina, mentioned in this section, correspond to the characteristics of the alumina before carrying out step a) of the preparation process according to the invention.

The support is an alumina, that is to say that the support comprises at least 95%, preferably at least 98% and particularly preferably at least 99% by weight of alumina, relative to the weight of the support. The alumina generally exhibits a crystallographic structure of the δ-, γ- or θ-alumina type, alone or as a mixture.

The alumina support may comprise impurities such as oxides of metals from groups IIA, IIIB, IVB, IIB, IIIA and IVA according to the CAS classification, preferably silica, titanium dioxide, zirconium dioxide, zinc oxide, magnesium oxide and calcium oxide, or else alkali metals, preferably lithium, sodium or potassium, and/or alkaline earth metals, preferably magnesium, calcium, strontium or barium, or else sulfur.

The specific surface area of the alumina is generally between 10 m²/g and 400 m²/g, preferably between 30 m²/g and 350 m²/g, more preferably between 50 m²/g and 300 m²/g.

The pore volume of the alumina is generally between 0.1 ml/g and 1.2 ml/g, preferably between 0.3 ml/g and 0.9 ml/g, and very preferably between 0.5 ml/g and 0.9 ml/g.

The median mesopore diameter is advantageously between 3 and 25 nm, preferably between 6 and 20 nm and particularly preferably between 8 and 18 nm.

### 5. Selective Hydrogenation Process

Another subject of the present invention is a process for the selective hydrogenation of polyunsaturated compounds containing at least 2 carbon atoms per molecule, such as diolefins and/or acetylenics and/or alkenylaromatics, also known as styrenics, contained in a hydrocarbon feedstock having a final boiling point below or equal to 300° C., which process being carried out at a temperature of between 0° C. and 300° C., at a pressure of between 0.1 and 10 MPa, at a hydrogen/(polyunsaturated compounds to be hydrogenated) mole ratio of between 0.1 and 10 and at an hourly space velocity of between 0.1 and 200 h⁻¹ when the process is carried out in the liquid phase, or at a hydrogen/(polyunsaturated compounds to be hydrogenated) mole ratio of between 0.5 and 1000 and at an hourly space velocity of between 100 and 40 000 h⁻¹ when the process is carried out in the gas phase, in the presence of a catalyst obtained by the preparation process as described above in the description.

Monounsaturated organic compounds, such as, for example, ethylene and propylene, are at the root of the manufacture of polymers, of plastics and of other chemicals having added value. These compounds are obtained from natural gas, from naphtha or from gas oil which have been treated by steam cracking or catalytic cracking processes. These processes are carried out at high temperature and produce, in addition to the desired monounsaturated compounds, polyunsaturated organic compounds, such as acetylene, propadiene and methylacetylene (or propyne), 1,2-butadiene and 1,3-butadiene, vinylacetylene and ethylacetylene, and other polyunsaturated compounds, the boiling point of which corresponds to the C5+ cut (hydrocarbon compounds having at least 5 carbon atoms), in particular diolefinic or styrene or indene compounds. These polyunsaturated compounds are highly reactive and result in side reactions in the polymerization units. It is thus necessary to remove them before making economic use of these fractions.

Selective hydrogenation is the main treatment developed to specifically remove undesirable polyunsaturated compounds from these hydrocarbon feedstocks. It makes possible the conversion of polyunsaturated compounds to the corresponding alkenes or aromatics while avoiding their complete saturation and thus the formation of the corresponding alkanes or naphthenes. In the case of steam cracking gasolines used as feedstock, the selective hydrogenation also makes it possible to selectively hydrogenate the alkenylaromatics to give aromatics while avoiding the hydrogenation of the aromatic rings.

The hydrocarbon feedstock treated in the selective hydrogenation process has a final boiling point of less than or equal to 300° C. and contains at least 2 carbon atoms per molecule and comprises at least one polyunsaturated compound. The term “polyunsaturated compounds” is understood to mean compounds comprising at least one acetylenic function and/or at least one diene function and/or at least one alkenylaromatic function.

More particularly, the feedstock is selected from the group consisting of a C2 steam cracking cut, a C2-C3 steam cracking cut, a C3 steam cracking cut, a C4 steam cracking cut, a C5 steam cracking cut and a steam cracking gasoline, also known as pyrolysis gasoline or C5+ cut.

The C2 steam cracking cut, advantageously used for the implementation of the selective hydrogenation process according to the invention, exhibits, for example, the following composition: between 40% and 95% by weight of ethylene and of the order of 0.1% to 5% by weight of acetylene, the remainder being essentially ethane and methane. In some C2 steam cracking cuts, between 0.1% and 1% by weight of C3 compounds can also be present.

The C3 steam cracking cut, advantageously used for the implementation of the selective hydrogenation process according to the invention, exhibits, for example, the following mean composition: of the order of 90% by weight of propylene and of the order of 1% to 8% by weight of propadiene and of methylacetylene, the remainder being essentially propane. In some C3 cuts, between 0.1% and 2% by weight of C2 compounds and of C4 compounds can also be present.

A C2-C3 cut can also advantageously be used for the implementation of the selective hydrogenation process according to the invention. It exhibits, for example, the following composition: of the order of 0.1% to 5% by weight of acetylene, of the order of 0.1% to 3% by weight of propadiene and of methylacetylene, of the order of 30% by weight of ethylene and of the order of 5% by weight of propylene, the remainder being essentially methane, ethane and propane. This feedstock can also contain between 0.1% and 2% by weight of C4 compounds.

The C4 steam cracking cut, advantageously used for the implementation of the selective hydrogenation process according to the invention, exhibits, for example, the following mean composition by weight: 1% by weight of butane, 46.5% by weight of butene, 51% by weight of butadiene, 1.3% by weight of vinylacetylene and 0.2% by weight of butyne. In some C4 cuts, between 0.1% and 2% by weight of C3 compounds and of C5 compounds can also be present.

The C5 steam cracking cut, advantageously used for the implementation of the selective hydrogenation process according to the invention, exhibits, for example, the following composition: 21% by weight of pentanes, 45% by weight of pentenes and 34% by weight of pentadienes.

The steam cracking gasoline or pyrolysis gasoline, advantageously used for the implementation of the selective hydrogenation process according to the invention, corresponds to a hydrocarbon cut, the boiling point of which is generally between 0 and 300° C., preferably between 1° and 250° C. The polyunsaturated hydrocarbons to be hydrogenated present in said steam cracking gasoline are in particular diolefin compounds (butadiene, isoprene, cyclopentadiene, and the like), styrene compounds (styrene, α-methylstyrene, and the like) and indene compounds (indene, and the like). The steam cracking gasoline generally comprises the C5-C12 cut with traces of C3, C4, C13, C14 and C15 (for example between 0.1% and 3% by weight for each of these cuts). For example, a feedstock formed of pyrolysis gasoline generally has a composition as follows: 5% to 30% by weight of saturated compounds (paraffins and naphthenes), 40% to 80% by weight of aromatic compounds, 5% to 20% by weight of mono-olefins, 5% to 40% by weight of diolefins and 1% to 20% by weight of alkenylaromatic compounds, the combined compounds forming 100%. It also contains from 0 to 1000 ppm by weight of sulfur, preferably from 0 to 500 ppm by weight of sulfur.

Preferably, the polyunsaturated hydrocarbon feedstock treated in accordance with the selective hydrogenation process according to the invention is a C2 steam cracking cut or a C2-C3 steam cracking cut or a steam cracking gasoline.

The selective hydrogenation process according to the invention is targeted at removing said polyunsaturated hydrocarbons present in said feedstock to be hydrogenated without hydrogenating the monounsaturated hydrocarbons. For example, when said feedstock is a C2 cut, the selective hydrogenation process is targeted at selectively hydrogenating acetylene. When said feedstock is a C3 cut, the selective hydrogenation process is targeted at selectively hydrogenating propadiene and methylacetylene. In the case of a C4 cut, the aim is to remove butadiene, vinylacetylene (VAC) and butyne; in the case of a C5 cut, the aim is to remove the pentadienes. When said feedstock is a steam cracking gasoline, the selective hydrogenation process is targeted at selectively hydrogenating said polyunsaturated hydrocarbons present in said feedstock to be treated so that the diolefin compounds are partially hydrogenated to give mono-olefins and so that the styrene and indene compounds are partially hydrogenated to give corresponding aromatic compounds while avoiding the hydrogenation of the aromatic rings.

The technological implementation of the selective hydrogenation process is, for example, carried out by injection, in upflow or downflow mode, of the polyunsaturated hydrocarbon feedstock and of the hydrogen into at least one fixed bed reactor. Said reactor can be of isothermal type or of adiabatic type. An adiabatic reactor is preferred. The polyunsaturated hydrocarbon feedstock can advantageously be diluted by one or more reinjection(s) of the effluent, resulting from said reactor where the selective hydrogenation reaction takes place, at various points of the reactor, located between the inlet and the outlet of the reactor, in order to limit the temperature gradient in the reactor. The technological implementation of the selective hydrogenation process according to the invention can also advantageously be carried out by the implantation of at least said supported catalyst in a reactive distillation column or in reactors-exchangers or in a slurry-type reactor. The stream of hydrogen can be introduced at the same time as the feedstock to be hydrogenated and/or at one or more different points of the reactor.

The selective hydrogenation of the C2, C2-C₃, C3, C4, C5 and C5+ steam cracking cuts can be carried out in the gas phase or in the liquid phase, preferably in the liquid phase for the C3, C4, C5 and C5+ cuts and in the gas phase for the C2 and C2-C3 cuts. A liquid-phase reaction makes it possible to lower the energy cost and to increase the cycle period of the catalyst.

In general, the selective hydrogenation of a hydrocarbon feedstock containing polyunsaturated compounds containing at least 2 carbon atoms per molecule and having a final boiling point below or equal to 300° C. is carried out at a temperature of between 0° C. and 300° C., at a pressure of between 0.1 and 10 MPa, at a hydrogen/(polyunsaturated compounds to be hydrogenated) mole ratio of between 0.1 and 10 and at an hourly space velocity (defined as the ratio of the volume flow rate of feedstock to the volume of catalyst) of between 0.1 and 200 h⁻¹ for a process carried out in the liquid phase, or at a hydrogen/(polyunsaturated compounds to be hydrogenated) mole ratio of between 0.5 and 1000 and at an hourly space velocity of between 100 and 40 000 h⁻¹ for a process carried out in the gas phase.

In one embodiment according to the invention, when a selective hydrogenation process is carried out wherein the feedstock is a steam cracking gasoline comprising polyunsaturated compounds, the (hydrogen)/(polyunsaturated compounds to be hydrogenated) mole ratio is generally between 0.5 and 10, preferably between 0.7 and 5.0 and more preferably still between 1.0 and 2.0, the temperature is between 0° C. and 200° C., preferably between 20° C. and 200° C. and more preferably still between 30° C. and 180° C., the hourly space velocity (HSV) is generally between 0.5 and 100 h⁻¹, preferably between 1 and 50 h⁻¹, and the pressure is generally between 0.3 and 8.0 MPa, preferably between 1.0 and 7.0 MPa and more preferably still between 1.5 and 4.0 MPa.

More preferentially, a selective hydrogenation process is carried out wherein the feedstock is a steam cracking gasoline comprising polyunsaturated compounds, the hydrogen/(polyunsaturated compounds to be hydrogenated) mole ratio is between 0.7 and 5.0, the temperature is between 20° C. and 200° C., the hourly space velocity (HSV) is generally between 1 and 50 h⁻¹ and the pressure is between 1.0 and 7.0 MPa.

More preferentially still, a selective hydrogenation process is carried out wherein the feedstock is a steam cracking gasoline comprising polyunsaturated compounds, the hydrogen/(polyunsaturated compounds to be hydrogenated) mole ratio is between 1.0 and 2.0, the temperature is between 30° C. and 180° C., the hourly space velocity (HSV) is generally between 1 and 50 h⁻¹ and the pressure is between 1.5 and 4.0 MPa.

The hydrogen flow rate is adjusted in order to have available a sufficient amount thereof to theoretically hydrogenate all of the polyunsaturated compounds and to maintain an excess of hydrogen at the reactor outlet.

In another embodiment according to the invention, when a selective hydrogenation process is carried out wherein the feedstock is a C2 steam cracking cut and/or a C2-C3 steam cracking cut comprising polyunsaturated compounds, the (hydrogen)/(polyunsaturated compounds to be hydrogenated) mole ratio is generally between 0.5 and 1000, preferably between 0.7 and 800, the temperature is between 0° C. and 300° C., preferably between 15° C. and 280° C., the hourly space velocity (HSV) is generally between 100 and 40 000 h⁻¹, preferably between 500 and 30 000 h⁻¹, and the pressure is generally between 0.1 and 6.0 MPa, preferably between 0.2 and 5.0 MPa.

### 6. Process for the Hydrogenation of Aromatics

Another subject of the present invention is a process for the hydrogenation of at least one aromatic or polyaromatic compound contained in a hydrocarbon feedstock having a final boiling point below or equal to 650° C., generally between 20° C. and 650° C., and preferably between 20° C. and 450° C. Said hydrocarbon feedstock containing at least one aromatic or polyaromatic compound can be chosen from the following petroleum or petrochemical fractions: the reformate from catalytic reforming, kerosene, light gas oil, heavy gas oil, cracking distillates, such as FCC recycle oil, coking unit gas oil or hydrocracking distillates.

The content of aromatic or polyaromatic compounds contained in the hydrocarbon feedstock treated in the hydrogenation process according to the invention is generally between 0.1% and 80% by weight, preferably between 1% and 50% by weight and particularly preferably between 2% and 35% by weight, the percentage being based on the total weight of the hydrocarbon feedstock. The aromatic compounds present in said hydrocarbon feedstock are, for example, benzene or alkylaromatics, such as toluene, ethylbenzene, o-xylene, m-xylene or p-xylene, or also aromatics having several aromatic rings (polyaromatics), such as naphthalene.

The sulfur or chlorine content of the feedstock is generally less than 5000 ppm by weight of sulfur or chlorine, preferably less than 100 ppm by weight and particularly preferably less than 10 ppm by weight.

The technological implementation of the process for the hydrogenation of aromatic or polyaromatic compounds is, for example, carried out by injection, as upflow or downflow, of the hydrocarbon feedstock and of the hydrogen into at least one fixed bed reactor. Said reactor can be of isothermal type or of adiabatic type. An adiabatic reactor is preferred. The hydrocarbon feedstock can advantageously be diluted by one or more reinjection(s) of the effluent, resulting from said reactor where the reaction for the hydrogenation of the aromatics takes place, at various points of the reactor, located between the inlet and the outlet of the reactor, in order to limit the temperature gradient in the reactor. The technological implementation of the process for the hydrogenation of the aromatics according to the invention may also advantageously be carried out by the implantation of at least said supported catalyst in a reactive distillation column or in reactors-exchangers or in a slurry-type reactor. The stream of hydrogen can be introduced at the same time as the feedstock to be hydrogenated and/or at one or more different points of the reactor.

The hydrogenation of the aromatic or polyaromatic compounds can be carried out in the gas phase or in the liquid phase, preferably in the liquid phase. Generally, the hydrogenation of the aromatic or polyaromatic compounds is carried out at a temperature of between 30° C. and 350° C., preferably between 50° C. and 325° C., at a pressure of between 0.1 and 20 MPa, preferably between 0.5 and 10 MPa, at a hydrogen/(aromatic compounds to be hydrogenated) mole ratio between 0.1 and 10 and at an hourly space velocity of between 0.05 and 50 h⁻¹, preferably between 0.1 and 10 h⁻¹, of a hydrocarbon feedstock containing aromatic or polyaromatic compounds and having a final boiling point below or equal to 650° C., generally between 20° C. and 650° C., and preferably between 20° C. and 450° C.

The hydrogen flow rate is adjusted in order to have available a sufficient amount thereof to theoretically hydrogenate all of the aromatic compounds and to maintain an excess of hydrogen at the reactor outlet.

The conversion of the aromatic or polyaromatic compounds is generally greater than 20 mol %, preferably greater than 40 mol %, more preferably greater than 80 mol % and particularly preferably greater than 90 mol % of the aromatic or polyaromatic compounds contained in the hydrocarbon feedstock. The conversion is calculated by dividing the difference between the total moles of the aromatic or polyaromatic compounds in the hydrocarbon feedstock and in the product by the total moles of the aromatic or polyaromatic compounds in the hydrocarbon feedstock.

According to a specific variant of the process according to the invention, a process for the hydrogenation of the benzene of a hydrocarbon feedstock, such as the reformate resulting from a catalytic reforming unit, is carried out. The benzene content in said hydrocarbon feedstock is generally between 0.1% and 40% by weight, preferably between 0.5% and 35% by weight and particularly preferably between 2% and 30% by weight, the percentage by weight being based on the total weight of the hydrocarbon feedstock.

The sulfur or chlorine content of the feedstock is generally less than 10 ppm by weight of sulfur or chlorine respectively and preferably less than 2 ppm by weight.

The hydrogenation of the benzene contained in the hydrocarbon feedstock can be carried out in the gas phase or in the liquid phase, preferably in the liquid phase. When it is carried out in the liquid phase, a solvent can be present, such as cyclohexane, heptane or octane. Generally, the hydrogenation of the benzene is carried out at a temperature of between 3° and 250° C., preferably between 5° and 200° C. and more preferably between 8° and 180° C., at a pressure of between 0.1 and 10 MPa, preferably between 0.5 and 4 MPa, at a hydrogen/(benzene) mole ratio between 0.1 and 10 and at an hourly space velocity of between 0.05 and 50 h⁻¹, preferably between 0.5 and 10 h⁻¹.

The conversion of the benzene is generally greater than 50 mol %, preferably greater than 80 mol %, more preferably greater than 90 mol % and particularly preferably greater than 98 mol %. The invention will now be illustrated via the examples below which are in no way limiting.

### EXAMPLES

For all the catalysts mentioned in the examples mentioned below, the support is an alumina A having a specific surface area of 180 m²/g, a pore volume of 0.7 ml/g and a median mesopore diameter of 10 nm.

### Example 1: Preparation of an Aqueous Solution S1 of First Ni Precursor with a First Organic Compound

The aqueous solution S1 is prepared by dissolving 58 g of nickel nitrate (NiNO₃ supplied by Strem Chemicals®) and 14.35 g of malonic acid (CAS 141-82-2; supplied by Fluka®) in a volume of 42 ml of distilled water. The solution is heated to 60° C. to facilitate the dissolution of the nickel nitrate. The additive/Ni mole ratio is fixed at 0.4 mol/mol. The solution S1 is obtained.

### Example 2: Preparation of an Aqueous Solution S2 of Precursors of the NiCu Alloy (5% by Weight of Ni) but without Second Organic Compound

The aqueous solution of precursors of the NiCu alloy (solution S2) used for the preparation of the catalysts containing NiCu is prepared by dissolving 14.5 g of nickel nitrate (NiNO₃, supplier Strem Chemicals®) in a volume of 13 ml of distilled water. A solution, the Ni concentration of which is 116.6 g of Ni per liter of solution, is obtained. The copper nitrate precursor is then added in order to have an Ni/Cu mole ratio of 1.

The solution S2 is obtained. It makes it possible to introduce the precursors of the NiCu alloy with a weight content of Ni relative to the final catalyst of 5% by weight relative to the total weight of the catalyst.

### Example 3: Preparation of an Aqueous Solution S3 of the Precursors of the NiCu Alloy and a Second Organic Compound (5% by Weight of Ni)

The aqueous solution of precursors of the NiCu alloy (solution S3) used for the preparation of the catalysts containing NiCu is prepared by dissolving 14.5 g of nickel nitrate (NiNO₃, supplier Strem Chemicals®) in a volume of 13 ml of distilled water. A solution, the Ni concentration of which is 116.6 g of Ni per liter of solution, is obtained. The copper nitrate precursor is then added in order to have an Ni/Cu mole ratio of 1. Malonic acid is also added to the solution in order to have an additive/Ni mole ratio of 0.90 mol/mol.

The solution S3 is obtained. It makes it possible to introduce the precursors of the NiCu alloy with a weight content of Ni relative to the final catalyst of 5% by weight relative to the total weight of the catalyst.

### Example 4: Preparation of a Catalyst a (not in Accordance with the Invention-No Second Organic Compound)

10 g of alumina A are dry impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. Next 10 ml of the solution S2 prepared in example 2 is dry impregnated by adding it dropwise. The solid thus obtained is subsequently dried in an oven for 12 hours at 120° C. and then calcined under a stream of dry air of 1 l/h/g of catalyst at 450° C. for 2 hours.

Catalyst A containing 24% by weight of the nickel element relative to the total weight of the catalyst (including 5% by weight of nickel element in the NiCu alloy) is obtained. The characteristics of the catalyst A thus obtained are given in table 1 below.

### Example 5: Preparation of a Catalyst B (not in Accordance with the Invention-No Second Organic Compound)

10 g of alumina A are dry impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. This intermediate catalyst is then dry re-impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. Next 10 ml of the solution S2 prepared in example 2 is dry impregnated by adding it dropwise. The solid thus obtained is subsequently dried in an oven for 12 hours at 120° C. and then calcined under a stream of dry air of 1 l/h/g of catalyst at 450° C. for 2 hours.

Catalyst B containing 27% by weight of the nickel element relative to the total weight of the catalyst (including 5% by weight of nickel element in the NiCu alloy) is obtained. The characteristics of the catalyst B thus obtained are given in table 1 below.

### Example 6: Preparation of a Catalyst C (in Accordance with the Invention)

10 g of alumina A are dry impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. Next 10 ml of the solution S3 prepared in example 3 is dry impregnated by adding it dropwise. The mole ratio between the 2^{nd} organic compound/Ni (NiCu solution, solution S2) and the 1st organic compound/Ni mole ratio (active Ni solution S1) is 2.25. The solid thus obtained is subsequently dried in an oven for 12 hours at 120° C. and then calcined under a stream of dry air of 1 l/h/g of catalyst at 450° C. for 2 hours.

Catalyst C containing 24% by weight of the nickel element relative to the total weight of the catalyst (including 5% by weight of nickel element in the NiCu alloy) is obtained. The characteristics of the catalyst C thus obtained are given in table 1 below.

### Example 7: Preparation of a Catalyst D (in Accordance with the Invention-Double Impregnation)

10 g of alumina A are dry impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. This intermediate catalyst is then dry re-impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. Then, 10 ml of the solution S3 prepared in example 3 is dry impregnated by adding it dropwise. The mole ratio between the 2nd organic compound/Ni (NiCu solution, S2 solution) and the 1st organic compound/Ni mole ratio (active Ni solution S1) is 2.25. The solid thus obtained is subsequently dried in an oven for 12 hours at 120° C. and then calcined under a stream of dry air of 1 l/h/g of catalyst at 450° C. for 2 hours.

Catalyst D containing 27% by weight of the nickel element relative to the total weight of the catalyst (including 5% by weight of nickel element in the NiCu alloy) is obtained. The characteristics of the catalyst D thus obtained are given in table 1 below.

### Example 8: Preparation of a Catalyst E (not in Accordance with the Invention-No NiCu and No Second Organic Compound)

10 g of alumina A are dry impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours. This intermediate catalyst is then dry re-impregnated with 7.1 ml of the solution S1. The catalyst precursor obtained is subsequently dried in an oven at 120° C. for 12 hours and then calcined under a stream of dry air of 1 l/h/g of catalyst at 450° C. for 2 hours.

The catalyst E containing 22% by weight of the element nickel, relative to the total weight of the catalyst, is obtained. The characteristics of the catalyst E thus obtained are given in table 1 below.

TABLE 1

| | Second organic | Ni° (% by | Particle size |
|---|---|---|---|
| Catalyst | compound | weight)* | (nm) |
| A (not in accordance | no | 19 | 6 |
| with the invention) | | | |
| B (not in accordance | no | 22 | 9 |
| with the invention) | | | |
| C (in accordance | yes | 19 | 2.5 |
| with the invention) | | | |
| D (in accordance | yes | 22 | 3 |
| with the invention) | | | |
| E (not in accordance | no (no NiCu) | 22 | 5 |
| with the invention) | | | |

*Ni° excluding NiCu alloy

### Example 9: Characterization

The amount of alloy obtained after the calcining step then reduction step was determined by X-ray diffraction (XRD) analysis on samples of the catalyst in powder form.

The amount of nickel in metallic form obtained after the reduction step was determined by X-ray diffraction (XRD) analysis on samples of catalyst in powder form. Between the reduction step and throughout the duration of the characterization by XRD, the catalysts are never returned to the open air. The diffraction patterns are obtained by radiocrystallographic analysis by means of a diffractometer using the conventional powder method with copper Kα1 radiation

$$( λ = 1.5406 Å ) .$$

The degree of reduction was calculated by calculating the area of the line of Ni⁰ located around 52°2θ, on all of the diffractograms of each sample of catalyst analyzed, then by subtracting the signal present as soon as ambient temperature is reached under the line at 52°, which is due to alumina.

Table 2 below collates the degrees of reduction or else the content of nickel metal Ni⁰ (expressed as % by weight relative to the total weight of “active” Ni which does not form the alloy) for all the catalysts A to E characterized by XRD after a reduction step at 170° C. for 190 minutes under a hydrogen stream. These values were also compared with the degree of reduction obtained for catalyst E (Ni alone) after a conventional reduction step (that is to say at a temperature of 400° C. for 15 hours under a hydrogen stream).

Alumina in delta and theta form and large CuO and NiO lines are detected at ambient temperature on all the copper- and nickel-containing catalysts, after calcination.

A line corresponding to the alloy in Ni_{0.76}Cu_{0.24} form is moreover detected after reduction.

In order to evaluate the degree of reducibility and therefore the formation of Ni⁰, the area of the line of Ni⁰ located around 52°2θ is measured, on all the diffractograms, by subtracting the signal present as soon as ambient temperature is reached under the line at 52°, which is due to the alumina. It is thus possible to determine the relative percentage of Ni⁰ crystallized after reduction.

TABLE 2

| | | | Particle | Percentage of Ni° |
|---|---|---|---|---|
| | Final | Ni/ | size | alone (XRD) after |
| Catalyst | reduction | Cu | (nm) | reduction (%) |
| A (not in accordance | 170° C., | yes | 6 | 90 |
| with the invention) | 190 min | | | |
| B (not in accordance | 170° C., | yes | 9 | 92 |
| with the invention) | 190 min | | | |
| C (in accordance | 170° C., | yes | 2.5 | 90 |
| with the invention) | 190 min | | | |
| D (in accordance | 170° C., | yes | 3 | 90 |
| with the invention) | 190 min | | | |
| E (not in accordance | 170° C., | no | 4 | 0* |
| with the invention) | 190 min | | | |
| E (not in accordance | 400° C., | no | 4 | 80 |
| with the invention) | 15 h | | | |

*Nickel in NiO form

For catalyst E (22% Ni alone/alumina), the degree of reducibility of nickel is 0% after exactly the same reduction treatment under hydrogen as for catalysts A, B, C, D. It is necessary to reduce catalyst E at 400° C. in order to have a reduction of the nickel oxide to Ni⁰ of about 80%.

### Example 10: Catalytic Tests: Performance in Selective Hydrogenation of a Mixture Containing Styrene and Isoprene (AHYD1)

Catalysts A to E described in the examples above are tested with regard to the reaction for the selective hydrogenation of a mixture containing styrene and isoprene.

The composition of the feedstock to be selectively hydrogenated is as follows: 8 wt % styrene (supplier Sigma Aldrich®, purity 99%), 8 wt % isoprene (supplier Sigma Aldrich®, purity 99%) and 84 wt % n-heptane (solvent) (supplier VWR®, purity>99% Chromanorm HPLC). This composition corresponds to the initial composition of the reaction mixture. This mixture of model molecules is representative of a pyrolysis gasoline.

The selective hydrogenation reaction is carried out in a 500 ml stainless steel autoclave which is provided with a magnetically-driven mechanical stirrer and which is able to operate under a maximum pressure of 100 bar (10 MPa) and temperatures of between 5° C. and 200° C. 214 ml of n-heptane (supplied by VWR®, purity>99% Chromanorm HPLC) and an amount of 3 ml of catalyst are added to an autoclave. The autoclave is closed and purged. The autoclave is then pressurized under 35 bar (3.5 MPa) of hydrogen. The catalyst is first reduced in situ, at 170° C. for 90 minutes under a hydrogen stream of 1 l/h/g (temperature rise gradient of 1° C./min) for catalysts A to E (which corresponds here to step f) of the preparation process according to the invention according to one embodiment). The autoclave is then brought to the test temperature equal to 30° C. At time t=0, approximately 30 g of a mixture containing styrene, isoprene, n-heptane, pentanethiol and thiophene are introduced into the autoclave. The reaction mixture then has the composition described above and stirring is started at 1600 rpm. The pressure is kept constant at 35 bar (3.5 MPa) in the autoclave using a storage cylinder located upstream of the reactor.

Another test was carried out for catalyst E, but with a catalyst reduction temperature of 400° C. for 15 hours.

The progress of the reaction is monitored by taking samples from the reaction medium at regular time intervals: the styrene is hydrogenated to give ethylbenzene, without hydrogenation of the aromatic ring, and the isoprene is hydrogenated to give methylbutenes. If the reaction is prolonged for longer than necessary, the methylbutenes are in their turn hydrogenated to give isopentane. The hydrogen consumption is also monitored over time by the decrease in pressure in a storage cylinder located upstream of the reactor. The catalytic activity is expressed in moles of H₂ consumed per minute and per gram of Ni.

The catalytic activities measured for catalysts A to E are reported in table 3 below. They are related to the catalytic activity (A_{HYD1}) measured for catalyst E prepared under conventional reduction conditions (at a temperature of 400° C. for 15 hours under a hydrogen stream).

### Example 11: Catalytic Tests: Performance in Hydrogenation of Toluene (A_{HYD2})

Catalysts A to E described in the above examples are also tested with regard to the reaction for the hydrogenation of toluene. The selective hydrogenation reaction is carried out in the same autoclave as that described in example 10.

214 ml of n-heptane (supplied by VWR®, purity>99% Chromanorm HPLC) and an amount of 3 ml of catalyst are added to an autoclave. The autoclave is closed and purged. The autoclave is then pressurized under 35 bar (3.5 MPa) of hydrogen. The catalyst is first reduced in situ, at 170° C. for 90 minutes under a hydrogen stream of 1 l/h/g (temperature rise gradient of 1° C./min) for catalysts A to E (which corresponds here to step f) of the preparation process according to the invention according to one embodiment). After addition of 216 ml of n-heptane (supplier VWR®, purity>99% Chromanorm HPLC), the autoclave is closed, purged, then pressurized under 35 bar (3.5 MPa) of hydrogen and brought to the test temperature, equal to 80° C. At time t=0, approximately 26 g of toluene (supplier SDS®, purity>99.8%) are introduced into the autoclave (the initial composition of the reaction mixture is then 6 wt % toluene/94 wt % n-heptane) and stirring is started at 1600 rpm. The pressure is kept constant at 35 bar (3.5 MPa) in the autoclave using a storage cylinder located upstream of the reactor.

The progress of the reaction is monitored by taking samples from the reaction medium at regular time intervals: the toluene is completely hydrogenated to give methylcyclohexane. The hydrogen consumption is also monitored over time by the decrease in pressure in a storage cylinder located upstream of the reactor. The catalytic activity is expressed in moles of H₂ consumed per minute and per gram of Ni.

The catalytic activities measured for catalysts A to E are reported in table 3 below. They are related to the catalytic activity (A_{HYD2}) measured for catalyst E prepared under conventional reduction conditions (at a temperature of 400° C. for 15 hours under a hydrogen stream).

TABLE 3

| Catalyst | Ni content (%) | A_{HYD1} (%) | A_{HYD2} (%) |
|---|---|---|---|
| A (not in accordance | 27 | 75 | 75 |
| with the invention) | | | |
| B (not in accordance | 27 | 76 | 80 |
| with the invention) | | | |
| C (in accordance | 24 | 160 | 170 |
| with the invention) | | | |
| D (in accordance | 27 | 180 | 195 |
| with the invention) | | | |
| E (not in accordance | 22 | <1 | <1 |
| with the invention) | | | |
| E (not in accordance | 22 | 100 | 100 |
| with the invention) | | | |

These results clearly show a significant improvement in the A_{HYD1} and A_{HYD2} performances of the catalysts C and D obtained by the preparation process according to the invention, compared to catalysts A, B and E not in accordance with the invention. For catalysts A and B, the nickel oxide is 90% reduced at 170° C. and has particles which have grown during the step of impregnation of NiCu without the presence of the second organic compound. Catalyst E has a reduced activity due to the absence of NiCu and therefore a virtually zero reducibility of the NiO at 170° C. Catalysts C and D retain small nickel particles due to the addition of malonic acid (second organic compound) during the post-treatment addition of NiCu compared with the addition of the precursor of the nickel active phase.

1. A process for preparing a catalyst comprising nickel and copper, in a proportion of 1% to 50% by weight of nickel element relative to the total weight of the catalyst, and in a proportion of 0.5% to 15% by weight of copper element relative to the total weight of the catalyst, and a porous alumina support, the size of the nickel particles in the catalyst, measured in oxide form, being less than 5 nm, the process comprising:
a) bringing the alumina support into contact with at least one solution containing at least one first nickel precursor and at least one first organic compound comprising at least one carboxylic acid function to obtain a first catalyst precursor;
b) drying the first catalyst precursor obtained at the end of a) at a temperature of less than 250° C., and then calcining the dried first catalyst precursor at a temperature of between 250° C. and 550° C. to obtain a calcined catalyst precursor;
c) bringing the calcined catalyst precursor obtained at the end of b) into contact with at least one solution containing at least one second nickel precursor, at least one copper precursor, and at least one second organic compound comprising at least one carboxylic acid function to obtain a second catalyst precursor; and
d) drying the second catalyst precursor obtained at the end of c) at a temperature of less than 250° C.

2. The process as claimed in claim 1, wherein the mole ratio between said organic compound introduced in a) and the nickel element also introduced in a) is between 0.01 and 5.0 mol/mol.

3. The process as claimed in claim 1, wherein the mole ratio between said organic compound introduced in c) and the nickel element also introduced in c) is between 0.02 and 5 mol/mol.

4. The process as claimed in claim 1, wherein the mole ratio between the nickel introduced during steps a) and c) and the copper introduced in step c) is between 0.5 and 5 mol/mol.

5. The process as claimed in claim 1, wherein the ratio between the mole ratio between the second organic compound and the nickel introduced in c) and the mole ratio between the first organic compound and the nickel introduced in a) is greater than 1.5.

6. The process as claimed in claim 1, wherein a) and b) are carried out at least twice before carrying out c).

7. The process as claimed in claim 1, wherein the first organic compound of a) and the second organic compound of c) are chosen from oxalic acid, malonic acid, glycolic acid, lactic acid, tartronic acid, citric acid, tartaric acid, pyruvic acid, and levulinic acid.

8. The process as claimed in claim 1, wherein the first organic compound of a) and the second organic compound of c) are identical.

9. The process as claimed in claim 1, wherein the copper precursor is chosen from copper acetate, copper acetylacetonate, copper nitrate, copper sulfate, copper chloride, copper bromide, copper iodide, and copper fluoride.

10. The process as claimed in claim 1, wherein the first nickel precursor and/or the second nickel precursor are/is nickel nitrate, nickel chloride, nickel acetate, or nickel hydroxycarbonate.

11. The process as claimed in claim 1, further comprising e) wherein the catalyst obtained at the end of d) is calcined at a temperature of between 250° C. and 550° C.

12. The process as claimed in claim 1, further comprising a step f) wherein the catalyst obtained at the end of d), optionally obtained at the end of e), is reduced by bringing said catalyst into contact with a reducing gas at a temperature of greater than or equal to 150° C. and less than 250° C.

13. A catalyst obtained via the process as claimed in claim 1, wherein said catalyst comprises nickel and copper, in a proportion of 1% to 50% by weight of nickel element relative to the total weight of the catalyst, and in a proportion of 0.5% to 15% by weight of copper element relative to the total weight of the catalyst, and a porous alumina support, the size of the nickel particles in the catalyst, measured in oxide form, being less than 5 nm.

14. A process for the selective hydrogenation of polyunsaturated compounds containing at least 2 carbon atoms per molecule, contained in a hydrocarbon feedstock having a final boiling point below or equal to 300° C., the process comprising:
performing selective hydrogenation of the polyunsaturated compounds in the presence of a catalyst as claimed in claim 13:
at a temperature of between 0° C. and 300° C., at a pressure of between 0.1 and 10 MPa, at a hydrogen/(polyunsaturated compounds to be hydrogenated) mole ratio of between 0.1 and 10 and at an hourly space velocity of between 0.1 and 200 h⁻¹ when the process is carried out in the liquid phase, or
at a temperature of between 0° C. and 300° C., at a pressure of between 0.1 and 10 MPa, at a hydrogen/(polyunsaturated compounds to be hydrogenated) mole ratio of between 0.5 and 1000 and at an hourly space velocity of between 100 and 40 000 h⁻¹ when the process is carried out in the gas phase.

15. A process for the hydrogenation of at least one aromatic or polyaromatic compound contained in a hydrocarbon feedstock having a final boiling point below or equal to 650° C., the process comprising:
performing hydrogenation of the at least one aromatic or polyaromatic compound in the gas phase or in the liquid phase, at a temperature of between 3° and 350° C., at a pressure of between 0.1 and 20 MPa, at a hydrogen/(aromatic compounds to be hydrogenated) mole ratio of between 0.1 and 10 and at an hourly space velocity of between 0.05 and 50 h⁻¹, in the presence of a catalyst as claimed in claim 13.
